(19) United States
(12) Patent Application Publication
(10) Pub. No.: US 2026/0165688 A1
(43) Pub. Date: Jun. 18, 2026

(54) Contact Allergy Fill System

(71) Applicants: Ty L. Prince, Knoxville, TN (US); Herman J. Novak, Maryville, TN (US)

(72) Inventors: Ty L. Prince, Knoxville, TN (US); Herman J. Novak, Maryville, TN (US)

(21) Appl. No.: 18/983,695

(22) Filed: Dec. 17, 2024

(51) Int. Cl. A61B 10/00; A61B 5/00

(52) U.S. Cl. CPC A61B 10/0035; A61B 5/411

(57) ABSTRACT

A syringe for holding and selectively releasing a desired amount of allergen, the syringe including electronic elements that at least one of detect, record, and transmit information in regard to the allergen, wherein the electronic elements include at least one of a sensor cap that fits on a dispensing end of a body of the syringe, and a sensor pack that fits along the body of the syringe.

### FIELD

This invention relates to the field of allergy treatment. More particularly, this invention relates to tracking, dispensing, dosing, reading, and analyzing allergens and reactions to allergens.

### INTRODUCTION

To avoid an allergic reaction, a person needs to know what they are allergic to. Skin testing is one way a health care provider can determine what allergenic material causes the allergic reaction. Such tests use extracts (a concentrated liquid form) of common allergens such as pollen, mold, dust mites, animal dander, foods, and many other materials. Once an allergen penetrates into the skin of the patient, it can trigger a reaction of some type. The presence of a reaction is how the health care provider determines a patient's allergies.

Health care providers tend to administer a large number of allergens when diagnosing a patient's allergies. Many allergen delivery systems use methods and devices that make it difficult for the health care provider to track the allergens that have been administered to the patient, or enable the allergen to be easily spilled or cross-contaminated, thereby possibly compromising or otherwise confounding the results of the testing procedure.

What is needed, therefore, is an allergen delivery system by which issues such as these are reduced, at least in part.

### SUMMARY

The above and other needs are met by an allergen pack for simultaneously holding more than one allergen dispensing unit, the allergen pack adapted such that a single end-effecter is operable to simultaneously dispense a desired identical amount of allergen from each of the allergen dispensing units in the allergen pack.

In some embodiments, the allergen dispensing units are syringes. In some embodiments, the allergen pack holds ten allergen dispensing units simultaneously. In some embodiments, the allergens in the allergen dispensing units are identified by at least one of a color, a number, a letter, a logo, a bar code, a Q-code, and a hatch pattern.

According to another aspect of the present disclosure, there is described an allergen patch that is adapted to simultaneously receive allergens into more than one associated dosing position, where a given one each of the allergens is associated with only a given one each of the dosing positions, and each dosing position is identified. In some embodiments, the allergen patch includes an adhesive layer on a side of the allergen patch that is adapted to receive the allergens, the adhesive layer adapted to provide temporary and selective adhesion between the allergen patch and skin of a patient at a dosing site at which the allergen patch is to be applied to the patient, such that the allergens transfer at least in part from the allergen patch to the dosing site on the patient's skin.

In some embodiments, the allergen patch includes a marking material on a side of the allergen patch that is adapted to receive the allergens, the marking material adapted to transfer to skin of a patient at a dosing site at which the allergen patch is to be applied to the patient, such that the marking material transfers at least in part from the allergen patch to the dosing site on the patient's skin. In some embodiments, the marking material is adapted to create a temporary mark on the patient's skin at the dosing site, and the mark is at least one of a number, a letter, a logo, a bar code, a Q code, and a hatch pattern. In some embodiments, the mark is adapted to indicate at least one of the dosing positions, the dosing allergens, the patient, a time at which the patient was dosed, the date on which the patient was dosed, and a health care provider who dosed the patient.

According to yet another aspect of the present disclosure, there is described a syringe for holding and selectively releasing a desired amount of allergen, the syringe including electronic elements that at least one of detect, record, and transmit information in regard to the allergen. In some embodiments, the electronic elements include at least one of a sensor cap that fits on a distal end of a plunger of the syringe, a sensor cap that fits on a dispensing end of a body of the syringe, a sensor pack that replaces a plunger of the syringe, and a sensor pack that fits along the body of the syringe. In some embodiments, the electronic elements are integrated into the formation of the syringe. In some embodiments, the electronic elements are adapted to be selectively added and removed from the syringe, as desired. In some embodiments, the information includes at least one of an identification of the allergen, a starting amount of allergen in the syringe, a dispensed amount of allergen from the syringe, a remaining amount of allergen in the syringe, and a temperature history of the allergen within the syringe.

Some embodiments include a cap that fits over a nozzle of the syringe, where the cap is at least partially formed of a thermoplastic resin with an orifice formed therein, such that when sufficient pressure is applied to the allergen within the syringe, the orifice opens and allows the allergen to flow from the nozzle, and when the pressure on the allergen is released, the orifice closes, thereby aiding in retention of the allergen within the syringe. In some embodiments, the cap is selectively removable from the syringe.

### BRIEF DESCRIPTION OF THE DRAWINGS

Further advantages of the invention are apparent by reference to the detailed description when considered in conjunction with the figures, which are not to scale so as to more clearly show the details, wherein like reference numbers indicate like elements throughout the several views, and wherein:

FIG. 1 is a depiction of allergen packs holding multiple allergen dispensing units, and associated allergen patches and allergen patch carriers, according to an embodiment of the present disclosure.

FIG. 2 is a depiction of a dispenser holding an allergen pack, an allergen patch, and an allergen patch carrier, according to an embodiment of the present disclosure.

FIG. 3 is a depiction of electronics associated with the dispenser and the allergen dispensing units, according to an embodiment of the present disclosure.

FIG. 4 is a depiction of electronics associated with an allergen patch carrier, according to an embodiment of the present disclosure.

FIG. 5 is a depiction of an allergen patch, according to an embodiment of the present disclosure.

FIG. 6 is a depiction of electronics associated with an allergen dispensing unit, according to a first embodiment of the present disclosure.

FIG. 7 is a depiction of electronics associated with an allergen dispensing unit, according to a second embodiment of the present disclosure.

FIG. 8 is a depiction of electronics associated with an allergen dispensing unit, according to a third embodiment of the present disclosure.

FIG. 9 is a depiction of an end cap for an allergen dispensing unit, according to an embodiment of the present disclosure.

FIG. 10 is a functional block diagram of an electronic device for an allergen testing system, according to an embodiment of the present disclosure.

### DESCRIPTION

### Overview

One goal of the system according to the present disclosure is to reduce human-induced mistakes and other errors in the allergen diagnosis process. Thus, the present system tends to standardize and systematize the processes that are used for the application, tracking, and analyzing of allergen materials and the reactions that they may cause.

According to the present disclosure, this is accomplished, in various embodiments that use all or a subset of the elements disclosed herein, including standardized groups or packs of allergens, which in some embodiments have some relationship one to another, which allergen packs are kept together as a group during storage, transport, and dispensing. The allergens are individually stored in separate syringes, which in some embodiments include at least one of mechanisms and electronics that track various properties and characteristics of the allergen.

These allergen packs are dispensed using a dispenser that dispenses all of the allergens in the pack simultaneously and at a metered amount. The allergens are delivered by the dispenser to patches that receive the metered amount of the allergens, which patches can be affixed onto the skin of the patient, and then removed after the allergens are transferred from the patch to the patient.

In some embodiments, an app or other device is used to record an image of the allergen delivery site on the patient. In various embodiments, the app performs some amount of manipulation and analysis of the image, in some embodiments requesting that additional images be captured under differing conditions. The app sends at least one of the image, the results of analyses in regard to the image, and other meta data to a server, for at least one of further analysis, diagnosis, prescription, and follow-up.

These various major elements of the system as introduced above are described in greater detail as given below.

With reference now to the drawings, there are depicted all of the claimed elements of the various embodiments, although all claimed embodiments might not be depicted in a single drawing. Thus, it is appreciated that not all embodiments include all of the elements as depicted, and that some embodiments include different combinations of the depicted elements. It is further appreciated that the various elements can all have many different configurations, and are not limited to just the configuration of a given element as depicted. As indicated above, the elements of the drawings as depicted are not to scale, even with respect one to another, and relative size or thickness of one element cannot be determined by the aspect ratios of that element or with reference to any dimension of another element.

### Allergen Packs

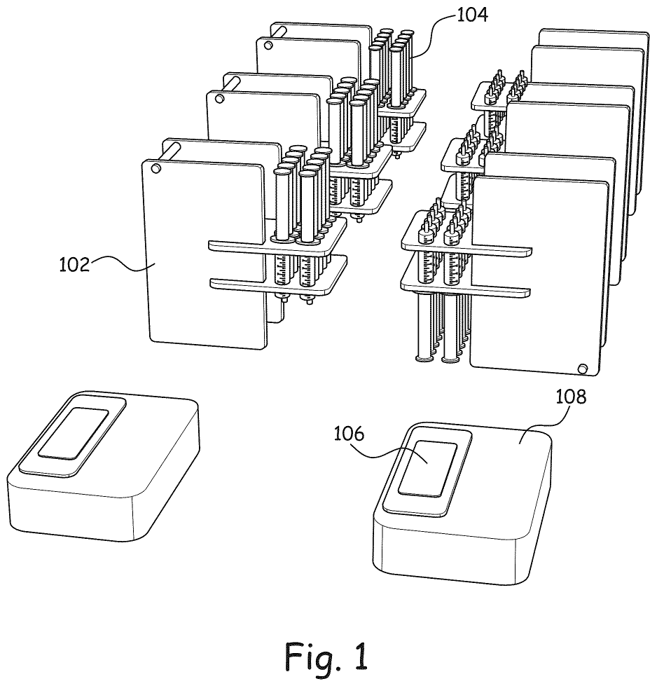

As depicted in FIG. 1, allergen packs 102 are assemblages of allergen dispensing units 104, such as syringes 104. The allergen packs 102 are, in some embodiments, groupings of similar types of allergens. For example, one allergen pack 102 might include a collection of allergens that are within a family of allergens. This might help narrow down which specific allergens within the family the patient is sensitive to. Another allergen pack 102 might contain one of each allergen from a wide variety of allergens, to determine generally what a patient might be sensitive to. Various other types of allergen collections within an allergen pack 102 are also contemplated.

In FIG. 1 as depicted, ten syringes 104 are included in each allergen pack 102. However, in various embodiments, a greater or lesser number of allergen dispensing units 104 might be included within the allergen pack 102, including a variety of different types of allergen dispensing units 104 within a given allergen pack 102. In some embodiments, the syringes 104 each have a label identifying the allergen contained therein. In some embodiments, the syringes 104 have a bar code, Q-code, or some other identifying indica or electronics for the allergen contents, or other information.

Also depicted in FIG. 1 are allergen patches 108, which are disposed upon patch carriers 106, both of which are introduced at this point, but described in greater detail hereafter. In some embodiments, at least one of the allergen patches 108 and the patch carriers 106 includes a characteristic that matches with a characteristic of an allergen pack 102.

The common characteristic is a method by which a given allergen patch 108 can be readily associated with a given allergen pack 102, such that the proper allergens are dispensed onto the allergen patch 108 to begin with, or by which it can be readily understood which allergens have already been dispensed onto a given allergen patch 108.

For example, the matching characteristic might be a predominant color used on each of the allergen pack 102 and the associated patch carrier 106. In another embodiment, the associated allergen pack 102 and allergen patch 108 might bear a common indicia, such at least one of a number, a letter, a combination of letters and numbers, a logo, a bar code, a Q code, and a hatch pattern. Other embodiments by which the allergen packs 102 can be associated with at least one of the allergen patches 108 and the patch carriers 106 are described in more detail hereinafter.

In some embodiments, a capping mechanism is included for capping the ends of all of the syringes 104 concurrently and immediately after the allergens therein have been dispensed.

### Dispenser

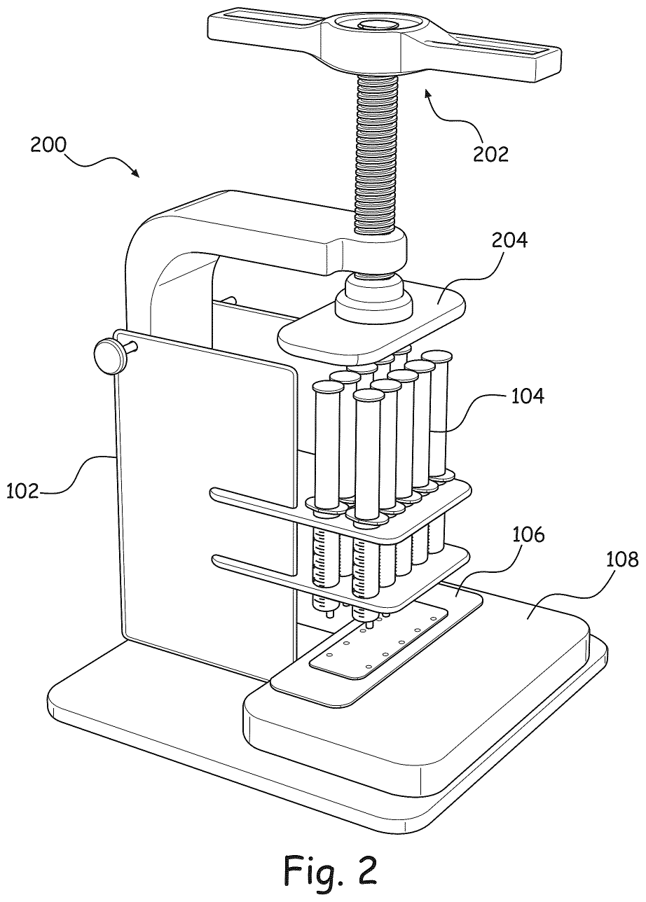

With reference now to FIG. 2, there is depicted a dispenser 200, which is used to simultaneously dispense a desired metered amount of allergen from each of the allergen dispensing units 104 an allergen 102 onto the allergen patch 106. As depicted, this is accomplished by affixing the allergen pack 102 in some manner to the dispenser 200, placing the patch carrier 108 in a precise location proximate the allergen dispensing units 104. A common end effector 204 is engaged with the allergen dispensing units 104, such that they all simultaneously release a desired metered amount of allergen onto precise and known locations on the allergen patch 106.

The end effector 204 is driven by an impeller 202, which takes different forms in different embodiments. For example, as depicted in FIG. 2, the impeller 202 can be as simple as a hand bar disposed on the first end of a lead screw, with the end effector 204 disposed on the second end of the lead screw. In other embodiments a hand crank can be used to drive a pinon that operates the lead screw. Another embodiment uses a lever to drive a plunger that is connected to the end effector 204. In still other embodiments, a motor is used to drive the mechanism by which the end effector 204 causes the allergen dispensing units 104 to dispense the allergens. Other various embodiments of the impellor 202 and end effector 204, as are known in the relevant art, are contemplated herein.

One aspect of the dispenser 200 in various embodiments is a known relationship between the operation of the impeller 202 and the amount of allergen that is dispensed by that operation. In this manner, a known movement of the impeller 202 dispenses a known and desired amount of allergen from each of the allergen dispensing units 104.

This can be accomplished by calibrating the movement of the impeller 202 with the characteristics of the allergen dispensing units 104. This calibrated relationship can be calculated for different types of allergen dispensing units 104, so that the same dispenser 200 can be used for different types of allergen dispensing units 104, and yet a known amount of allergen is able to be dispensed onto the allergen patch 106.

In the embodiment of a hand operated impeller 202 as depicted, notches on at least one of the lead screw and the portion of the dispenser 200 that engages the lead screw can provide tactile feedback for the rotation of the lead screw. The technician can sense and count the number of tactile bumps as the lead screw is rotated, and with a knowledge of the relationship between the tactile bumps and the allergen dispensing units 104, the amount of allergen dispensed can be carefully controlled. In another embodiment, the technician watches the movement of the plungers of the syringes 104 in relationship to indicia on the bodies of the syringes 104 to control the amount of allergen that is dispensed. Other embodiments are also contemplated herein.

### Smart Syringes

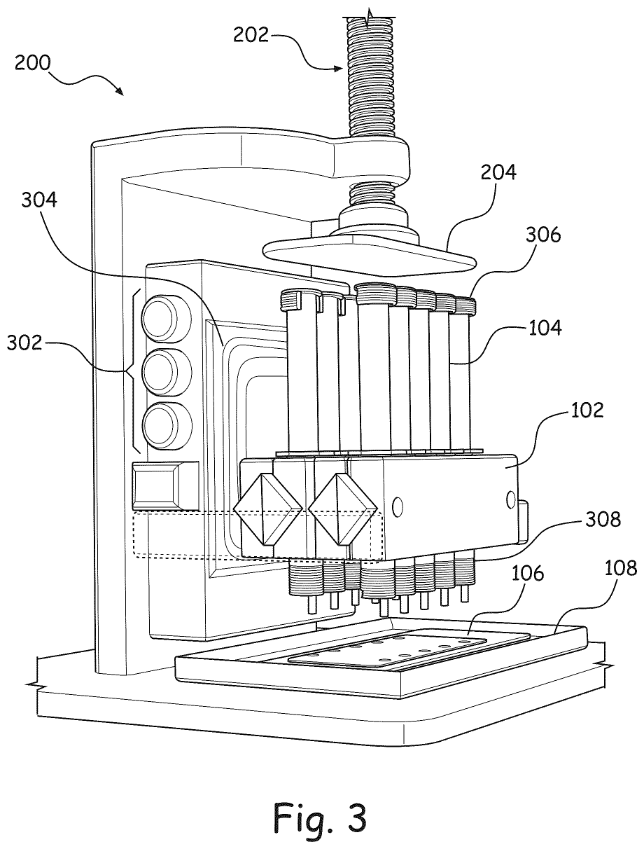

With reference now to FIG. 3, there is depicted various embodiments of the allergen dispensing units 104 and the dispenser 200. In this embodiment, the allergen dispensing units 104 are one type of so-called smart syringes 104, which include a base 306 and a cap 308, both of which include electronics of some sort, which react in some manner with at least one of each other and sensor 304 disposed in the dispenser 200. The interaction between the electronics of the base 306, cap 308, and sensor 304 are operative to determine one or more of a variety of different properties in regard to the allergens in the smart syringes 104, such as the amount of allergen dispensed from the smart syringes 104.

For example, in some embodiments the base 306 and cap 308 contain active RFID electronics, and the sensor 304 senses the relative positions of the bases 306 and the caps 308. Thus, as the end effector 204 presses down on the plungers of the smart syringes 104, the change in positions of the bases 306 relative to the caps 308 is detected by the sensor 304, and the known relationship between the relative change in position and the amount of allergen dispensed enables the dispenser 200 to dispense the desired quantity of allergen onto the allergen patch 106.

In various embodiments, the dispenser 200 performs some amount of storage and analysis of some or all of the data that it receives from various sources. In some embodiments, the dispenser 200 transmits some or all of the data that it receives, along with some or all of the results of the analyses that it optionally performs locally, to a remote device, such as a server that stores the data and analyses so received with other additional data.

Also depicted in FIG. 3 are user controls 302 for the operation of the dispenser 200, which are representative of a user interface, such as for starting, stopping, and pausing a dispensing cycle of the dispenser 200. Additionally, a different embodiment of the allergen pack 102 is depicted, wherein either one or two separate allergen packs 102 are selectively loaded into the dispenser 200. More detail in regard to this embodiment of the allergen packs 102 is provided hereinafter.

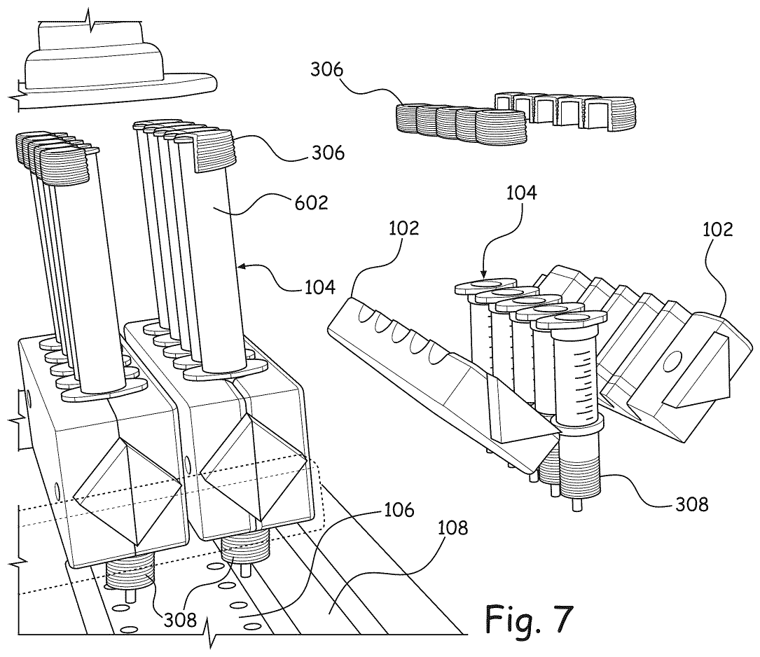

With reference now to FIG. 7, there is depicted additional detail in regard to the embodiments introduced in FIG. 3, including a depiction of one embodiment of a separable allergen pack 102.

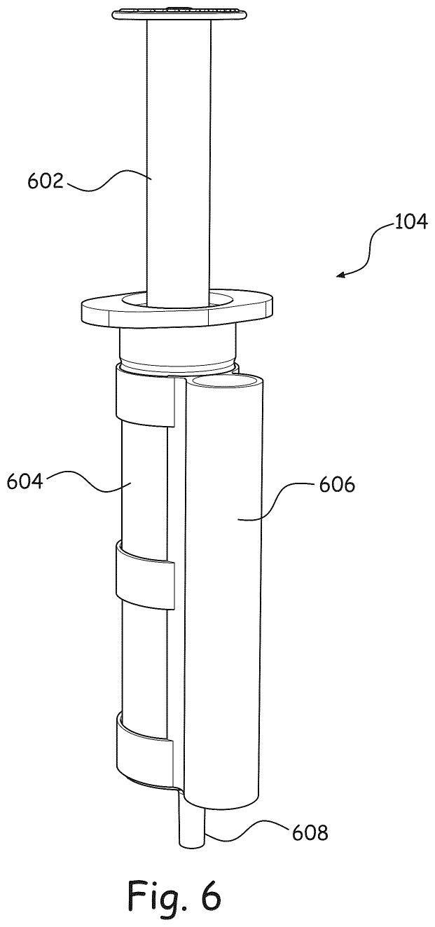

With reference now to FIG. 6, there is depicted another embodiment of the smart syringe 104, in which a sensor pack 606 is snapped or slid or otherwise releasably affixed to the body 604 of a standard syringe 104, turning it into a smart syringe 104. In some embodiments, as the plunger 602 is lowered into the body 604 of the smart syringe 104 and allergen is expelled from the nozzle 608, the sensor pack 606 detects at least one of the progress of the plunger 602 or the depletion of the allergen, thereby enabling a determination of the amount of the allergen that has been expelled from the smart syringe 104. In some embodiments, the plunger 602 is a specialized plunger 602 that is placed into a standard body 604, which specialized plunger 602 optionally functions in cooperation with the sensor pack 606 to detect at least one of the progress of the plunger 602 or the depletion of the allergen.

In some embodiments, additional electronics within the sensor pack 606 or the specialized plunger 602 transmit this information, either sua sponte or upon receipt of a signal to transmit such, to another device. In some embodiments, that other device is electronics within the dispenser 200. When the smart syringe 104 is empty, it can be refilled or it can be discarded after the sensor pack 606 and specialized plunger 602 are removed from the body 604, as applicable, with the sensor pack 606 and specialized plunger 602 free to be reused with another syringe body 604, in various embodiments.

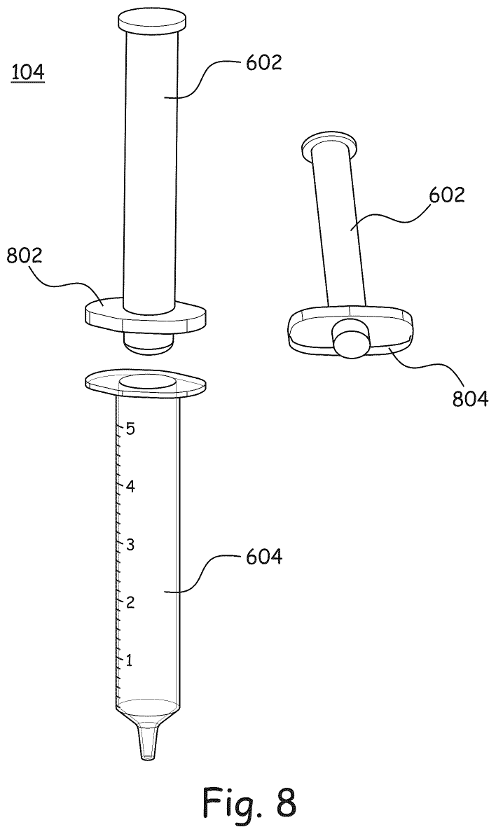

With reference now to FIG. 8, there is depicted another embodiment of the smart syringe 104, in which the electronics for converting a standard syringe 104 into a smart syringe 104 are wholly disposed within the plunger 602. In the embodiment as depicted, the plunger 602 is slidably retained by a collar 802 that snaps over the end of the body 604 of the syringe 104, which receives the end of the body 604 within a recess 804.

The electronics within the plunger 602 determine how much allergen has been dispensed from the smart syringe 104. This can be accomplished in a variety of different ways. For example, in some embodiments the electronics can sense the movement of the plunger 602 as it slides through the collar 802. In some embodiments, the electronics in the plunger 602 sense the amount of allergen remaining within the body 604 such of the smart syringe 104, such as by detecting a capacitance of the fluid. Other embodiments are also contemplated herein.

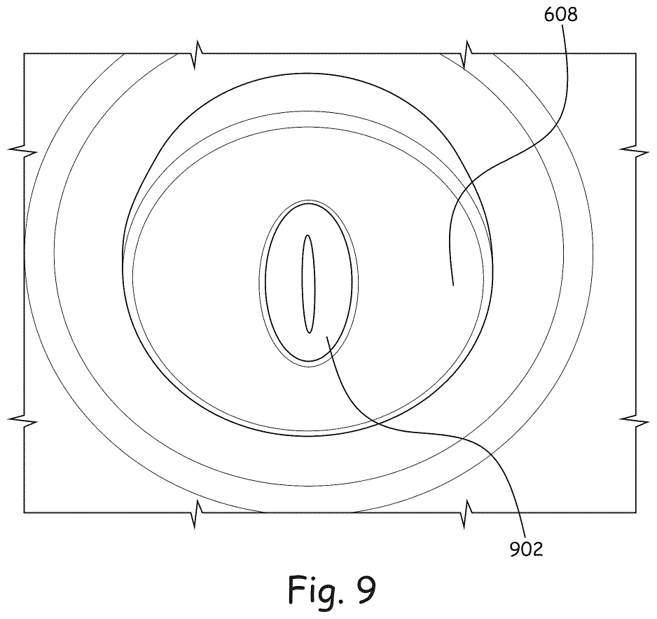

With reference now to FIG. 9, there is depicted an embodiment of a cap 902 that fits over the nozzle 608 of the syringe 104. The cap 902, in some embodiments, is at least partially formed of a thermoplastic resin with an orifice formed therein. When sufficient pressure is applied to the allergen within the syringe 104, the orifice opens and allows the allergen to flow from the nozzle 608. However, when the pressure on the allergen is released, such as by removing pressure on the plunger 602, the orifice closes, thereby aiding in the retention of the liquid allergen within the syringe 104. In some embodiments, these caps 902 are selectively removable from the syringe 104. In some embodiments, the caps 902 are disposed of when the syringe 104 has exhausted its supply of allergen. In some embodiments, the caps 902 are formed as a part of the caps 308, as described hereinabove in regard to FIG. 3.

In various embodiments, the smart syringe 104 detects a variety of parameters in regard to the use of the smart syringe 104, such as at least one of the ambient temperature, a history of the ambient temperature, the temperature of the allergen, a history of the allergen temperature, the amount of allergen dispensed, the time at which the allergen was dispensed, the rate at which the allergen was dispensed, and the amount of allergen remaining. This information is, in various embodiments, associated with other information such as at least one of the identification of the patient, the identification of the health care provider, the source of the allergen, the location at which the patient is dosed, and a history of the location of the smart syringe 104.

### Patch Carriers

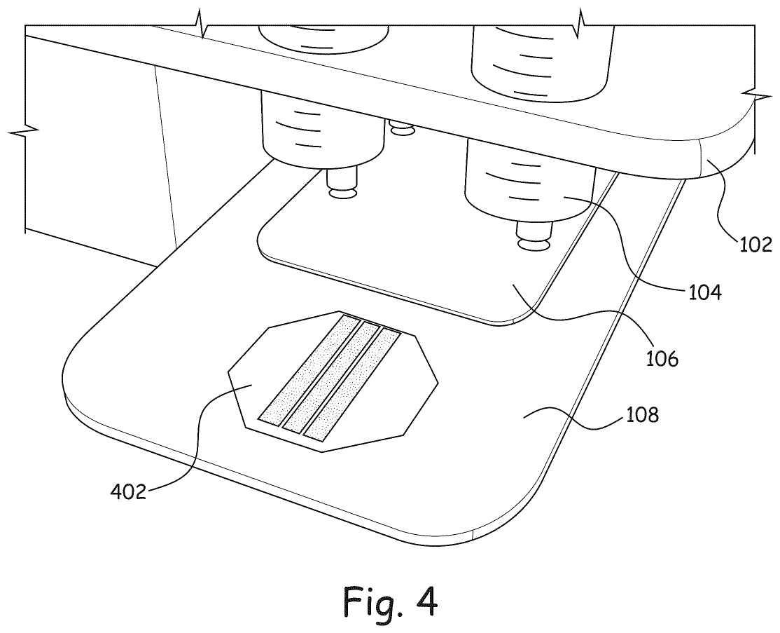

With reference now to FIG. 4, the embodiments that include electronics can be extended to the patch carriers 108, which can themselves include an electronic element 402, such as a smart RFID tag 402, that can identify to the sensor 304 at least one of the type of allergen patch 106 that is disposed on the patch carrier 108, and the type of allergens that should be dispensed onto the allergen patch 106. Other information in regard to at least one of the allergen patch 106 and the patch carrier 108 can also be encoded within the electronics 402, as desired, in various embodiments.

### Allergen Patches

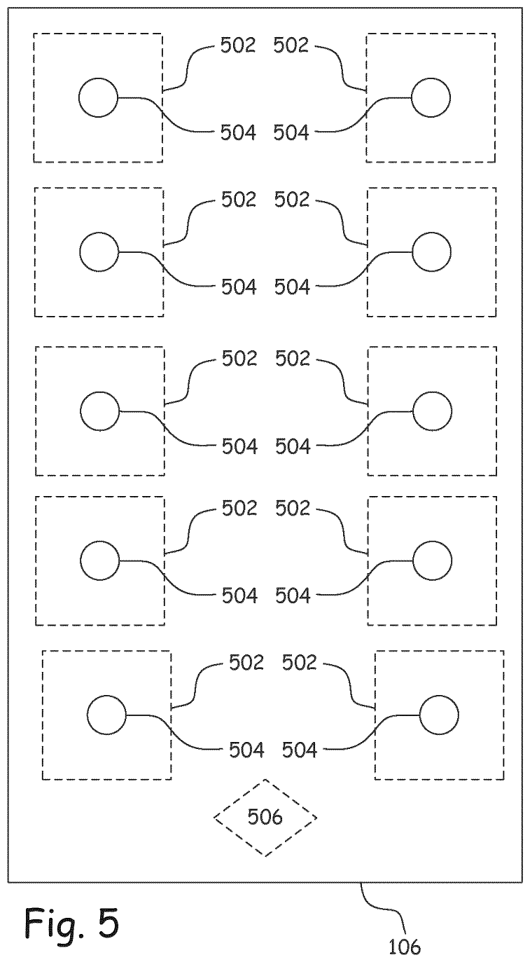

With reference now to FIG. 5, there is depicted a top plan view of an allergen patch 106 according to an embodiment of the present disclosure. In this embodiment, multiple allergen deposit sites 502 are marked on allergen patch 106, with the number of allergen deposit sites 502 corresponding to the number of allergen delivery units 104 in the associated allergen pack 102 that is used to dispense the allergens onto the allergen patch 106.

Also disposed on the allergen patch 106 are allergen wells 504, which are adapted to receive the dispensed allergens. In some embodiments, the allergen wells 504 have one or more characteristics that cause the allergens dispensed within the allergen wells 504 to tend to stay within the allergen wells 504, rather than spread out across the surface of the allergen patch 106. In some embodiments this characteristic is a recess in the allergen well 504 relative to the rest of the surface of the allergen patch 106, such that the allergen dispensed therein tends to remain with the recess of the allergen well 504.

In some embodiments, the characteristic of the allergen wells 504 is an absorbent material that is affixed to the allergen patch 106, such that the allergen dispensed into the allergen wells 504 remains within the absorbent material of the allergen wells 504. Other embodiments for having the allergens remain within the allergen wells 504 are also contemplated herein.

In some embodiments, boundary lines of the allergen deposit sites 502 are created on the allergen patch 106 using a material that transfers to the skin of the patient when the allergen-dosed allergen patch 106 is applied to the skin of the patient, in such a manner that the material temporarily stains the skin of the patient. For example, the boundary lines are printed using a transferrable ink or dye, such as henna, which transfers to the skin of the patient, identifying the allergen exposure sites on the patient, but which wears off over time so as to not permanently mark the patient's skin. In this manner, the allergen deposit sites 502 can be readily identified on the patient's skin, enabling ready identification of those sites in which a reaction to the allergen has occurred.

In some embodiments, some type of identifying indicia 506 is included on the allergen patch 106, where the identifying indicia 506 contains information in regard to at least one of the patient, the time and date on which the patient was dosed with the allergens, and the allergens with which the patient was dosed. Other information could also be included in the identifying indicia 506.

The identifying indicia 506 has different forms in different embodiments. In some embodiments, the identifying indicia 506 is at least one of a bar code, a Q code, a numeric code, and plain descriptive text. In some embodiments, the identifying indicia 506 is formed in the same manner as the boundary lines of the allergen deposit sites 502, so that a marking of the identifying indicia 506 is transferred to the patient's skin, and temporarily remains on the patient's skin for some length of time. In other embodiments, the information described herein is encoded in a series of at least one of notches, tabs, and slits, such as at one or more of the edges of the allergen patch 106. These and similar methods can also be used, in some embodiments, to indicate the various positions of the allergens (1-10, for example).

### Reader

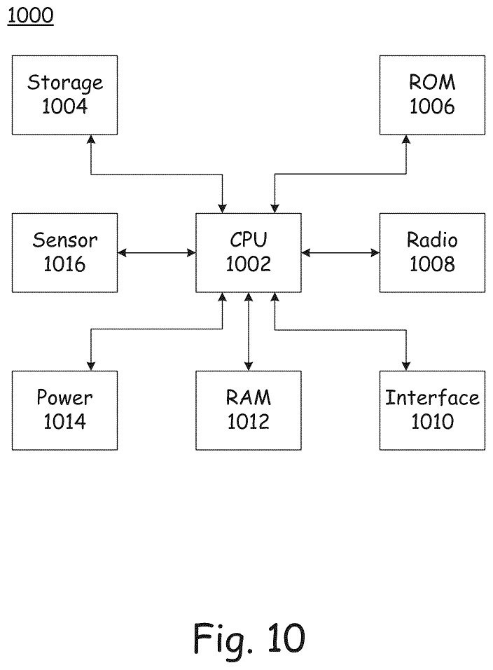

With reference now to FIG. 10, there is depicted a reader 1000 that is used in some embodiments to selectively detect and analyze the results of the application of the allergens to the patient, and which is used in some embodiments with the allergen patch 106 as described above.

As an overview, the reader 1000 includes in various embodiments of the electronics and programming to enable it to create and analyze an image of the allergen-dosed area on the skin of the patient, at one or more points in time, and detect, compare, and analyze the changes in the skin reactions produced by the various allergens so applied, and to perform the various other actions as described herein. The reader 1000 is at least one of a special purpose computing device, a tablet computer, a smart phone, a smart watch, a component level processor, an application specific integrated circuit, or some other computing device.

In some embodiments, the sensor 1016 is a camera that can create an image of the dosing site on the patient. In some embodiments, the radio 1008 enables data communication over a wireless connection, including at least one of RFID, Bluetooth, UWB, cellular, Wi-Fi, Zigbee, and Z-Wave.

In this embodiment, the reader 1000 is locally under the control of the central processing unit 1002, which controls and utilizes the other modules of the reader 1000 as described herein. As used herein, the word module refers to a combination of both software and hardware that performs one or more designated function. Thus, in different embodiments, various modules might share elements of the hardware as described herein, and in some embodiments might also share portions of the software that interact with the hardware. In some other embodiments, a given module might be spread across different computer platforms.

The reader 1000 as depicted in FIG. 10 includes, in some embodiments, a non-transitory, computer-readable, data storage medium module 1004 such as a flash drive, or some other relatively long-term data storage device. A read-only memory module 1006 contains, for example, basic operating instructions for the operation of the reader 1000. A radio module 1008 provides a gateway for the communication of data and instructions between the reader 1000 and other computing devices, networks, or data storage modules. An interface module 1010 includes, for example, keyboards, speakers, microphones, cameras, displays, and touchscreens, and provides means by which the user can interact with the reader 1000.

A random-access memory module 1012 provides short-term storage for data, such as image data, that is being buffered, analyzed, or manipulated and programming instructions for the operation of the reader 1000. A power module 1014 is also provided in various embodiments of the reader 1000. In some embodiments, the power module 1014 is a portable power supply, such as one or more batteries. In some embodiments the power module 1014 includes a renewable source, such as a solar panel or an inductive coil that are configured to provide power or recharge batteries.

In some embodiments the steps of the various functions as described herein are embodied in a computer language on a non-transitory, computer-readable, data storage medium that is readable by the reader 1000 of FIG. 10, and that enables the reader 1000 to implement the functions as described herein, such as a removable memory card.

In some embodiments the reader 1000 is to be used by the patient, such as in the patient's home, and be a device that is owned by the patient, such as the patient's cell phone. At a certain point in time, or at multiple points in time, the patient takes a picture of the allergen dosing site on the patient's skin, and uses the reader 1000 to upload the image so captured to the health care provider's server, or the server of a third party service provider.

For example, the patient can photograph the test area at home after two days, four days, and seven days, with no need to return to the health care provider's office. The reader 1000 and associated software identifies the patient and records the positive or negative results of the allergen tests. The data is sent to the cloud, or in other words, a remotely-accessed server. A rules-based or AI analysis system compares the test results and sends the results to the health care provider. The health care provider either accepts the results or changes them, if deemed necessary. This feedback loop thereby trains the analysis system.

In some embodiments, the dosing site includes the indicia 506 that is transferred from allergen patch 106 to the patient's skin. In this manner, in some embodiments, the image itself contains all the information that is needed by the health care provider to identify the source of the image. In other embodiments, information in regard to the source of the image is either manually entered into the reader 1000, such as by the patient herself, or automatically entered by the reader 1000, which source information is sent with the image as meta data to the health care provider.

### Automation

When the dispenser 200 is triggered by the movement of the end effecter 204, the dispenser 200 at least one of measures and transmits at least one of the amount of allergen dispensed, the allergen in the syringe 104, the temperature history of the allergen, the expiration date of allergen, an automatically generated order for more allergen when the syringe 104 is at least one of near a designated empty condition, and close to its expiration date, and the quantity of allergen remaining in the syringe 104. This data plus any additional desired data, such as data in regard to the patent, is sent to the cloud in some embodiments, for at least one of analysis and archival purposes.

As used herein, the phrase “at least one of A, B, and C” means all possible combinations of none or multiple instances of each of A, B, and C, but at least one A, or one B, or one C. For example, and without limitation: Ax1, Ax2+Bx1, Cx2, Ax1+Bx1+Cx1, Ax7+Bx12+Cx113. It does not mean Ax0+Bx0+Cx0.

The foregoing description of embodiments for this invention has been presented for purposes of illustration and description. It is not intended to be exhaustive or to limit the invention to the precise form disclosed. Obvious modifications or variations are possible in light of the above teachings. The embodiments are chosen and described in an effort to provide illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. (canceled)

2. (canceled)

3. (canceled)

4. (canceled)

5. (canceled)

6. (canceled)

7. (canceled)

8. (canceled)

9. (canceled)

10. A syringe for holding and selectively releasing a desired amount of allergen, the syringe including electronic elements that at least one of detect, record, and transmit information in regard to the allergen, wherein the electronic elements include a sensor pack that fits along the outside of the body of the syringe, wherein the sensor pack is adapted to be selectively added and removed from the syringe, as desired.

11. (canceled)

12. The syringe of claim 10, wherein the electronic elements are integrated into the syringe.

13. (canceled)

14. The syringe of claim 10, wherein the information includes at least one of an identification of the allergen, a starting amount of allergen in the syringe, a dispensed amount of allergen from the syringe, a remaining amount of allergen in the syringe, and a temperature history of the allergen within the syringe.

15. The syringe of claim 10, further comprising a cap that fits over a nozzle of the syringe, the cap at least partially formed of a thermoplastic resin with an orifice formed therein, such that when sufficient pressure is applied to the allergen within the syringe, the orifice opens and allows the allergen to flow from the nozzle, and when the pressure on the allergen is released, the orifice closes, thereby aiding in retention of the allergen within the syringe.

16. The syringe of claim 15, wherein the cap is selectively removable from the syringe.
